(19) United States
(12) Patent Application Publication
(10) Pub. No.: US 2016/0015262 A1
(43) Pub. Date: Jan. 21, 2016

(54) METHOD AND SYSTEM FOR IMPROVING VISION OF AN EYE WITH MACULAR DEGENERATION

(71) Applicants: David M. Lieberman, New York, NY (US); Jonathan Grierson, Abilene, TX (US)

(72) Inventors: David M. Lieberman, New York, NY (US); Jonathan Grierson, Abilene, TX (US)

(21) Appl. No.: 14/522,686

(22) Filed: Oct. 24, 2014

Related U.S. Application Data
(63) Continuation of application No. PCT/US11/26941, filed on Mar. 3, 2011.

(51) Int. Cl. A61B 3/00; G02C 7/02; A61F 2/16; A61B 3/107; A61F 9/008; A61F 2/14

(52) U.S. Cl. CPC A61B 3/0025; A61F 9/00802; A61F 2/142; A61F 2/1613; A61F 2/1602; A61B 3/107; G02C 7/02; G02C 7/027; A61F 2009/00872; A61F 2009/00882; G02C 2202/10

(57) ABSTRACT

Methods and apparatus are disclosed for diagnosing vision and improving vision, for example by reducing or eliminating the effects of macular degeneration, in a manner which does not interfere with the natural shape of the cornea or its orientation relative to the remainder of the eye, but which changes its surface curvature appropriately to achieve the required correction of vision. The focus of sub-regions of the cornea is adjusted so that different regions focus at a controlled distance about a reference axis. This can be accomplished by shaping the cornea (e.g. through ablation) or by applying an appropriate contact lens or other optical lens.

The present patent application is a continuation of International Application No. PCT/US2011/026941 filed Mar. 3, 2011, which was published in English under Publication No. WO 2011/109571 on Sep. 9, 2011, and which claimed the priority of U.S. Provisional Application No. 61/310,073. Each of the preceding documents is hereby incorporated by reference in its entirety.

### BACKGROUND OF THE INVENTION

The present invention relates generally to a method and system for diagnosing and improving the vision of an eye and, more particularly, to improvement of the vision of an eye with macular degeneration.

Macular degeneration is a progressive disease of the retina of the eye in which the light-sensing cells in the central area of vision (the macula) cease to function properly. The most common form of macular degeneration is age-related macular degeneration, and it is most common in people who are age 60 and over. In the early stages of the disease, there may be a slight loss of central vision, including a dark or blurry central spot (a central scotoma). As the disease progresses, central vision is increasingly lost, until it disappears entirely in the advanced stages. This disease is the leading cause of blindness in senior citizens. Approximately 15 million people in the United States have it, and approximately 2 million new cases are diagnosed annually.

The present invention provides for the improvement of vision in an eye with macular degeneration. It contemplates ablation procedures of the cornea and the provision of various types of corrective lenses, including contact lenses and spectacles.

Ophthalmologists model the cornea as a portion of an ellipsoid defined by orthogonal major and minor axes. Current surgical procedures for correcting visual acuity are typically directed at increasing or decreasing the surface curvature of the cornea, while making its shape more spherical, or conforming it to an “average” ellipse, or making corrections based on wavefront analysis.

In conjunction with modern corneal procedures, such as corneal ablation surgery, for clinical applications, and for contact lens design and manufacture, high resolution cameras are used to obtain a digitized array of discrete data points on the corneal surface. One system and camera which have been available for mapping the cornea is the PAR Corneal Topography System (PAR CTS) of PAR Vision Systems. The PAR CTS maps the corneal surface topology in three-dimensional Cartesian space, i.e., along x- and y-coordinates, as well as a depth (z) coordinate.

The “line-of-sight” is a straight line segment from a fixation point to the center of the entrance pupil. As described more fully in Mandell, “Locating the Corneal Sighting Center From Videokeratography,” J. Refractive Surgery, V. 11, pp. 253-259, July/August 1995), a light ray which is directed toward a point on the entrance pupil from a point of fixation will be refracted by the cornea and aqueous humor and pass through a corresponding point on the real pupil to eventually reach the retina.

The point on the cornea at which the line-of-sight intersects the corneal surface is the “optical center” or “sighting center” of the cornea. It is the primary reference point for refractive surgery in that it usually represents the center of the area to be ablated in photorefractive keratectomy. The line-of-sight has conventionally been programmed into a laser control system to govern corneal ablation surgery. However, some surgeons prefer to use the pupillary axis as a reference line. Experienced practitioners have employed various techniques for locating the sighting center. In one technique, the angle lambda is used to calculate the position of the sighting center relative to the pupillary (“optic”) axis. See Mandell, supra, which includes a detailed discussion of the angles kappa and lambda, the disclosure of which incorporated herein by reference as if set forth in its entirety herein.

In current LASIK corneal ablation procedures, a portion of the corneal surface or a surface under a flap is ablated. Gathered elevational data is used to direct an ablation device, such as a laser, so that the corneal surface can be selectively ablated to more closely approximate a spherical surface of appropriate radius about the line-of-sight, (or an “average” ellipse, or a wavefront fingerprint) within the ablation zone. The use of the line-of-sight as a reference line for the procedures may reduce myopia or otherwise correct a pre-surgical dysfunction or a visual abnormality. However, a more irregularly shaped cornea may result, which may exacerbate existing astigmatism or introduce astigmatism or spherical aberration in the treated eye. This will complicate any subsequent vision correction measures that need be taken. Also, any substantial surface irregularities which are produced can cause development of scar tissue or the local accumulation of tear deposits, either of which can adversely affect vision.

Implicit in the use of the-line-of sight or the pupillary axis as a reference axis for surgical procedures is the assumption that the cornea is symmetric about an axis extending along a radius of the eye. The cornea, however, is an “asymmetrically aspheric” surface. “Aspheric” means that the radius of curvature along any corneal “meridian” is not a constant (a “meridian” could be thought of as the curve formed by the intersection of the corneal surface and a plane containing a reference axis, such as the pupillary axis). Indeed, the corneal curvature tends to flatten progressively from the geometric center to the periphery. “Asymmetric” means that the corneal meridians do not exhibit symmetry about their centers. The degree to which the cornea is aspheric and/or asymmetrical varies from patient to patient and from eye to eye within the same person.

Analysis of clinical measurements in accordance with surface modeling techniques disclosed in U.S. Pat. No. 5,807,381 assigned to the assignee of the present patent application, reveals that the cornea exhibits a tilt, typically a forward and downward tilt, relative to the eye. This tilt may be as great as 6° and, on the average, is between 1° and 3°. Hence, a corneal ablation procedure which utilizes the line-of-sight or pupillary axis as a reference axis tends to over-ablate some portions of the cornea and under-ablate other potions of the cornea. At the same time, it changes the geometric relationship between the ablated cornea and the remainder of the eye. Thus, any ablation procedure which does not take into account the tilt of the cornea is not likely to achieve the desired shaping of the cornea and may therefore be unpredictable in its effect. Similarly, a contact lens design (or any other lens used to improve vision) which does not take into account the tilt cannot achieve optimum results.

Analysis of clinical measurements in accordance with the surface modeling techniques of U.S. Pat. No. 5,807,381 also reveals that the point on the surface of the cornea which is most distant from the reference plane of the PAR CTS (hereafter referred to as the HIGH point) is a far more effective reference point for corneal ablation and lens design than the center of the cornea or the pupillary center. Specifically, as demonstrated in U.S. Pat. No. 5,807,381 laser ablation about an axis passing through the HIGH point produces a much more regularly shaped cornea and removes less corneal material than the same operation performed about an axis close to the center of the eye, such as the pupillary axis.

Although incorporating corneal tilt and utilizing the HIGH point produce improved and more consistent results with corneal ablation surgery, there is still an excessively high degree of unpredictability. For example, analyses of clinical measurements have revealed that, in some eyes, the postoperative cornea begins to change shape a short time after corneal ablation surgery. Thus, a nearly perfectly spherical post-operative cornea of the type most commonly produced by conventional surgery will, over time, return to an aspheric, asymmetric shape.

Analysis of clinical measurements in accordance with the methods of U.S. Pat. No. 5,807,381, and International Application No. PCT/US03/1763 (published as W003/101341), the disclosures both of which are incorporated herein by reference in their entirety, raises questions about assumptions that have been made about the structure of the human cornea which are inherent in such well-known corneal analysis technologies as wave-front analysis and placido disc technology. In particular, it was found that, unlike other optical systems, the central portion of the cornea (for example, out to a 3 mm diameter) is not necessarily optically superior to substantially greater portions of the cornea (for example, out to a 7 mm diameter) in its ability to focus. The central portion of the cornea exhibits a great deal of focus scattering. That is, different regions on the cornea do not focus to the same point on a focal axis. Indeed, they do not even focus on the axis. This focus difference is most pronounced in the central portion of the cornea and decreases substantially at increasing diameters from the center.

As disclosed in PCT/US03/1763, vision can be improved by adjusting the focus of the cornea, referred to as “orthogonalizing”, so that different regions focus substantially to the same axis. This can be accomplished by shaping the cornea (e.g. through ablation) or by applying an appropriate corrective lens, effectively reducing radial and axial focus scatter. An additional benefit of orthogonalization was that presbyopia (defective near vision) was substantially reduced. That is, presbyopic patients fitted with orthogonalized contact lenses that did not have components that focused at different distances had improved near vision to the extent of not requiring reading glasses.

Subsequent experimentation has revealed that the symptoms of macular degeneration can be reduced through orthogonalization, but by doing so less than perfectly. In accordance with the present invention, orthogonalization is performed so as to produce a predetermined amount of imperfection in the orthogonalization. This will be referred to as “decentered orthogonalization.” The invention contemplates that light be delivered to the macula in patterns designed to avoid areas of the macula with “dead” light receptors.

### BRIEF DESCRIPTION OF THE DRAWINGS

The foregoing brief description, as well as other objects, features and advantages of the present invention will be understood more completely from the following detailed description of presently preferred embodiments, with reference being had to the accompanying drawings in which:

FIG. 1 is a block diagram illustrating a method for achieving vision correction in accordance with the present invention through either laser ablation of the cornea or an appropriately shaped lens;

FIG. 2 is a schematic diagram illustrating a plan view of a point cloud as obtained with a corneal image capture system;

FIG. 3 is a schematic plan view similar to FIG. 2 illustrating a plurality of splines and how they are connected through the data points of the point cloud;

FIG. 4 is a perspective view of a cornea matching surface illustrating how characterizing curves are constructed;

FIG. 5 is a diagram exemplifying the axial focus scatter of a cornea at a 3 millimeter diameter.

FIG. 6 illustrates the radial focus scatter corresponding to FIG. 5;

FIG. 7 is a diagram exemplifying the axial focus scatter of a cornea at a 5 millimeter diameter;

FIG. 8 illustrates the radial focus scatter corresponding to FIG. 7;

FIG. 9 is a diagram exemplifying the axial focus scatter of a cornea at a 7 millimeter diameter;

FIG. 10 illustrates the radial focus scatter corresponding to FIG. 9;

FIG. 11 illustrates a method for modifying the corneal model by orthogonalizing to the central axis;

FIG. 12 illustrates the concept of decentered orthogonalization; and

FIGS. 13-15 are plan views of the macula showing the 72 focus points P distributed in spiral, rose and dual rose patterns, respectively, on the anterior surface of the macula.

### DETAILED DESCRIPTION OF THE PREFERRED EMBODIMENTS

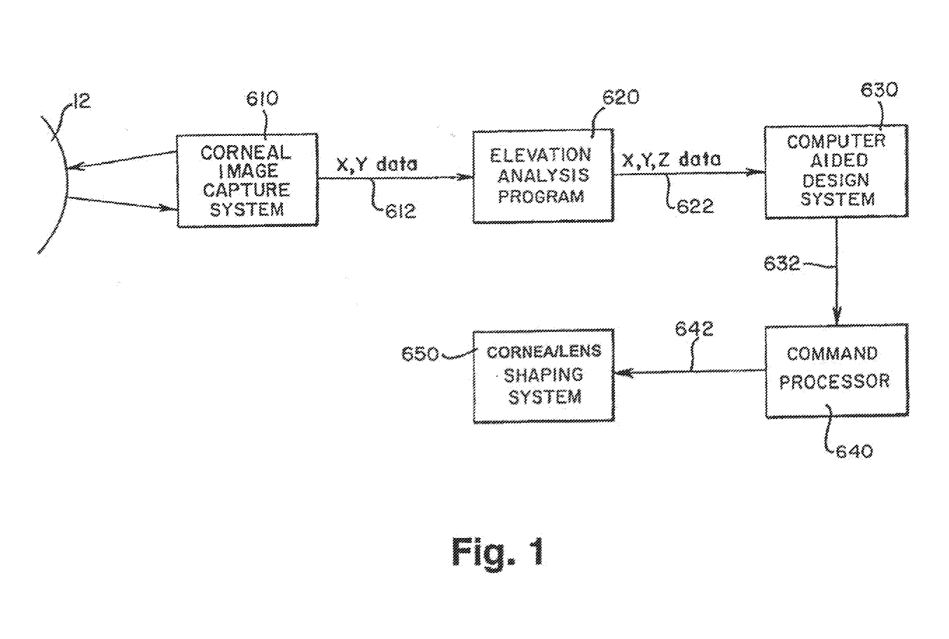

A process for achieving laser ablation of the cornea and contact lens shaping in accordance the present invention is illustrated in block diagram form in FIG. 1. The process makes use of a Corneal Image Capture System 610, an Elevation Analysis Program 620, a Computer Aided Design System 630, a Command Processor 640 and a Cornea Shaping System 650. The Corneal Image Capture System 610, in conjunction with the Elevation Analysis Program 620, generates a three dimensional topographic map of the cornea of the patient. The Computer Aided Design System 630 is used as an aid in editing or modifying the corneal topographic data, to create a surface model, and data relating to the model is sent to a Cornea Shaping System 650 via the Command Processor 640. The Command Processor 640 uses the topographic data describing the surface of the cornea to be shaped from the Computer Aided Design System 630 to generate a sequence of commands/control signals required by the Cornea/Lens Shaping System 650. The Cornea/Lens Shaping System 650 accepts, from the Command Processor 640, a sequence of commands that describe the three dimensional movements of the Cornea/Lens Shaping System (any coordinate system may be used; e.g., Cartesian, radial or spherical coordinates) to shape the cornea or machine (e.g. a lathe) manufacturing a contact lens.

The Corneal Image Capturing System 610 and the Elevation Analysis Program 620 are preferably components of the PAR® Corneal Topography System (“the PAR® System”), which is available from PAR Vision Systems. The Elevation Analysis Program 620 is a software program executed by a processor, for example an IBM™ compatible PC. Program 620 generates a third dimension element (a Z coordinate representing distance away from a reference plane inside the eye) for each of a plurality of sample points on the surface of the cornea measured by system 610. Each point is defined by its X-Y coordinates as mapped into the reference plane, and its Z coordinate is determined from brightness of the point. One method of calculating the elevation of each point, i.e., the Z coordinate, is by comparing the X-Y and brightness values measured from the patient's cornea 14 with the coordinates and brightness of some reference surface with known elevation, e.g., a sphere of a known radius. The reference values can be pre-stored.

The final output of the Elevation Analysis Program 620 is the X-Y-Z coordinates for a multiplicity of sample points, commonly known as a point cloud, on the surface of the cornea 14. It will be apparent to those skilled in the art that any method can be used that can generate X, Y, Z corneal data providing both location and elevation information for points on the corneal surface with the required accuracy. In the preferred embodiment about 1200 points are spaced in a grid pattern, as viewed in the X-Y plane, so the projections of the points into the X-Y plane are about 200 microns apart.

The X-Y-Z data output from the Elevation Analysis Program 620 can be formatted in any number of well-known machine-specific formats. In the preferred embodiment, the data are formatted in Data Exchange File (DXF) format, an industry standard format which is typically used for the inter-application transfer of data. A DXF file is an ASCII data file, which can be read by most computer aided design systems.

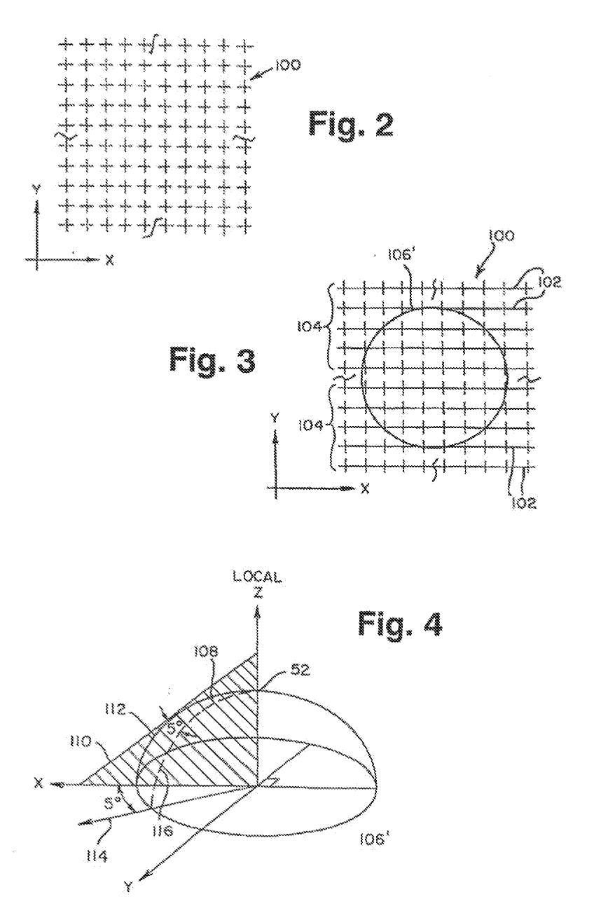

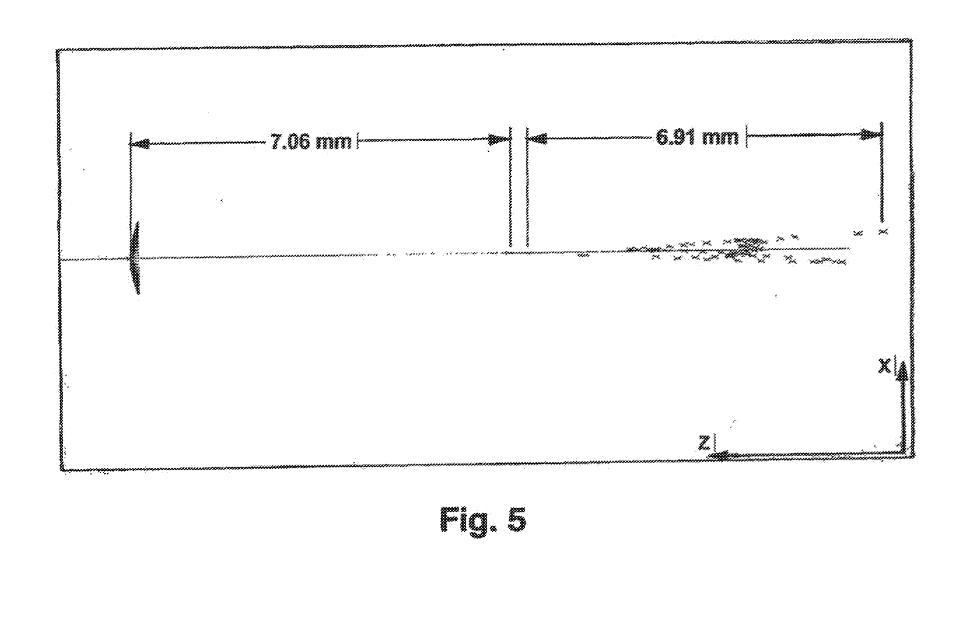

Referring now to FIGS. 2 and 3, a point cloud 100 is depicted as it would appear when viewing the reference plane along the Z-axis (i.e., as projected into the X-Y plane). Each point corresponds to a particular location on the patient's cornea. The data are usually generated from an approximately 10 mm×10 mm bounded area of the cornea, the working area. Thus, there may be as many as 50 rows of data points. A surface 108 (see FIG. 4) that models or matches the topography of the surface of the patient's cornea is generated by the computer aided design system 630 from the data points generated by the Elevation Analysis Program. In a preferred embodiment, Computer Aided Design System 630 is the Anvil 5000™ program which is available from Manufacturing Consulting Services of Scottsdale, Ariz.

Cornea matching surface 108 is preferably produced by first generating a plurality of splines 102, each defined by a plurality of the data points of the point cloud 100. The generation of a spline that intersects a plurality of data points (i.e., knot points) is, per se, known to those skilled in the art and can be accomplished by the Anvil 5QQQ™ program once the input data have been entered. For more information regarding the generation of a surface model, see U.S. Pat. No. 5,807,381, the disclosure of which is incorporated herein by reference in its entirety. In a preferred embodiment, the known non-uniform rational B-spline formula is used to generate the splines, but they could be generated by other well-known mathematical formulas for splines, such as the cubic spline formula or the rational uniform B-spline formula. As illustrated in FIG. 3, in a preferred embodiment, each of the splines 102 lies in a plane that is parallel to the X and Z axes and includes a row of points from the cloud 100 in FIG. 3.

Surface 108, which matches the corneal surface of the scanned eye, is then generated from splines 102. There are a number of well-known mathematical formulas that may be used to generate a surface from a plurality of splines 102. In the preferred embodiment, the well known nurb surface equation is used to generate a corneal surface from splines 102. In the embodiment, because the scanned area of the eye is approximately 10 mm×10 mm, approximately 50 splines 102 are created. As illustrated in FIG. 3, a skinned surface segment 104 is created for a small number (e.g., five) of the adjacent splines. Adjacent skinned surface segments 104 share a common border spline. Thus, about ten skinned surface segments are generated from the point cloud and are then merged together by the Anvil 5000™ program in a manner known to those skilled in the art, to produce one composite surface 108.

Neither the original data points, nor the knot points of splines 102 necessarily lie on-surface 108, owing to the mathematical generation of the surface when using the nurb surface equation formula. However, the surface 108 estimates those points within a predefined tolerance.

The HIGH point on the generated corneal matching surface 108 (i.e., the point having the greatest Z value) is determined. A cylinder 106 of a predetermined diameter is then projected onto the corneal matching surface 108 along an axis which is parallel to the Z-axis and passes through the HIGH point. Cylinder 106 preferably has a diameter of about 3 mm to about 8 mm, typically about 7 mm, and the closed contour formed by the intersection of cylinder 106 with surface 108 projects as a circle 106′ in the X-Y plane. On the matching surface 108, this contour defines the outer margin 26 of the working area of the cornea. The cornea is the most symmetric and spherical about the HIGH point and, therefore, provides the best optics at this point.

The outer margin 26 must fit within the point cloud, so that the surfaces of the cornea can be formed based on the measured corneal data. The computer aided design system 630 can then illustrate a default circle 106′ (in the X-Y plane) with respect to the point cloud, for example on a monitor screen, so that the operator can be assured that circle 106′ falls within the point cloud. Additionally, system 630 can be set up to determine if circle 106′ falls within point cloud 100 and, if it does not fall completely within point cloud 100, to alert the user to manipulate the circle (i.e., move the center point and/or change the radius of the circle) so that circle 106′ lies within the corneal data point cloud 100. In a worst case scenario, the eye should be rescanned if insufficient data is available from the scanned eye to ensure that the working area of the cornea will fit properly within the point cloud. Alternatively, the area of the point cloud can be made larger.

It is to be understood that circle 106′ is only a circle when viewed in the X-Y plane (i.e., looking along the Z-axis). Actually, the periphery 26 is approximately elliptical and lies in a plane which is tilted relative to the reference plane. A line Perpendicular to this tilted plane which passes through the HIGH point will be referred to as the “LOCAL Z-AXIS” or “tilted axis”, and the tilt of the tilted plane relative to the reference plane will be considered the tilt angle of the working area of the cornea.

The cornea is about 600 pm thick. In most corneal ablation procedures, less than 100 pm depth of cornea is ablated because there is virtually no risk of scarring with the type of lasers that are typically used. Beyond the 100 pm depth, there is a risk of scar-like imperfections. For example, 120 pm depth ablation is known to cause scarring. However, there exists the possibility that the risk of scarring for surface ablations may be reduced by drug therapy prior to or contemporaneous with the laser treatment. However, most of today's laser surgery does not cause scarring, as most procedures are under the LASIK flap. The fear in LASIK is ablating too deep wherein the residual bed is less than −250 pm. If the bed is less than this amount, structural failure can occur. The magnitude of the corneal undulations is typically about fifteen to twenty microns from the crest of a hill to the trough of a valley and may be as great as about thirty microns.

Surgical procedures performed in accordance with the present invention and optical lenses manufactured in accordance with the invention, in addition to relieving macular degeneration, will seek to correct the patient's vision in accordance with the required corrections established in a “refraction test.” When this test is performed, the patient sits in chair which is fitted with a special device called a “phoropter”, through which the patient looks at an eye chart approximately 20 feet away. As the patient looks into the phoropter, the doctor manipulates lenses of different strengths into view and, each time, asks the patient whether the chart appears more or less clear with the particular lenses in place. In practice, the doctor is able to vary the power or diopter correction about two orthogonal axes, as well as the degree of rotation of those axes about a Z-axis along the line-of-sight. The doctor continues to modify these three parameters until he achieves the optimum vision. The results of the refraction test are usually given in the form “a, b, c”, where “a” is the diopter correction at the first axis, “b” is the additional diopter correction required at the second, orthogonal axis, and “c” is the angle of rotation of the first axis relative to the horizontal. This form of information is given for each eye and is immediately useful in grinding a pair of lenses for eyeglasses.

For the purposes of the present invention, it is preferred to perform a modified form of refraction test. For this modified form of refraction test, the eye doctor adjusts the phoropter at a series of equally spaced angles, say every 15° from the horizontal, and obtains the optimum refraction at each angle. Typically, the more angles that are measured, the better the results. The manner of using the modified refraction test will be described in detail below.

There will now be described a technique for generating characterizing curves on surface 108, which will be useful below. A plane 110 is constructed which contains the LOCAL Z-AXIS (See FIG. 4). The intersection between plane 110 and surface 108 defines a first characterizing curve 112. Plane 110 is then rotated about the LOCAL Z-AXIS, for example by a 5° increment counterclockwise, as represented by line 114, where its intersection with surface 108 defines a second characterizing curve 116, which is illustrated as a dashed line in FIG. 4. This process continues at fixed rotational increments about the LOCAL Z-AXIS, for example every 5°, until plane 110 has swept 360°, to produce a complete set of characterizing curves (meridians), in this case seventy-two (360° % 5°).

Each of these characterizing curves is then estimated by a best-fit spherical (circular) arc. One manner of doing this is simply to select a circular arc which passes through three known points for each curve (e.g. the point at which it touches the contour 106′, the HIGH point, and that point which is halfway between those two points when viewed in projection along the local Z axis). Once the spherical arcs are generated, the focal point of a portion of the cornea represented by a circular arc can be estimated by the center of that arc. Techniques for locating the center of a spherical arc are well-known. The resulting set of arc centers then provides a representation of focus scattering.

For purposes of illustration, the preceding procedure was performed on the corneal model of a patient having 20/15 uncorrected visual acuity.

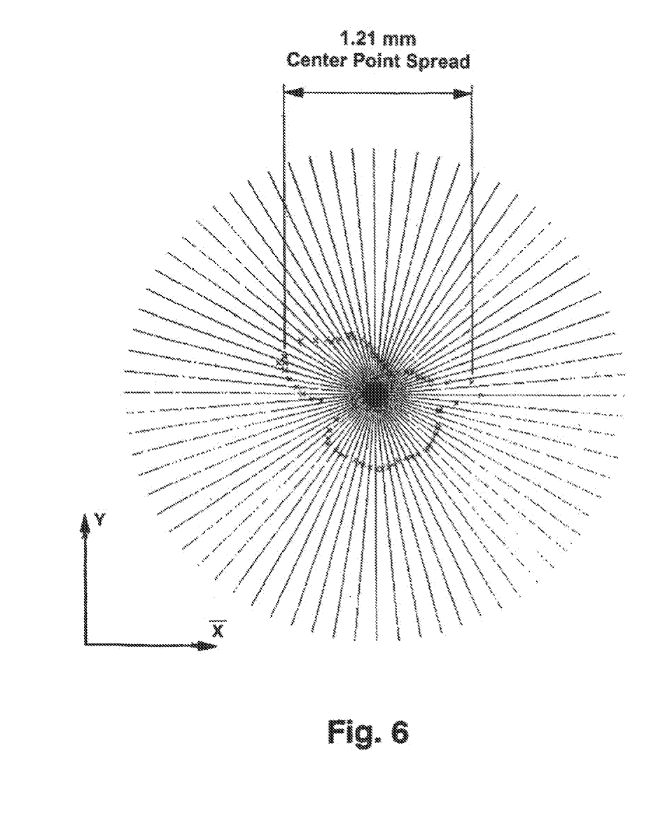

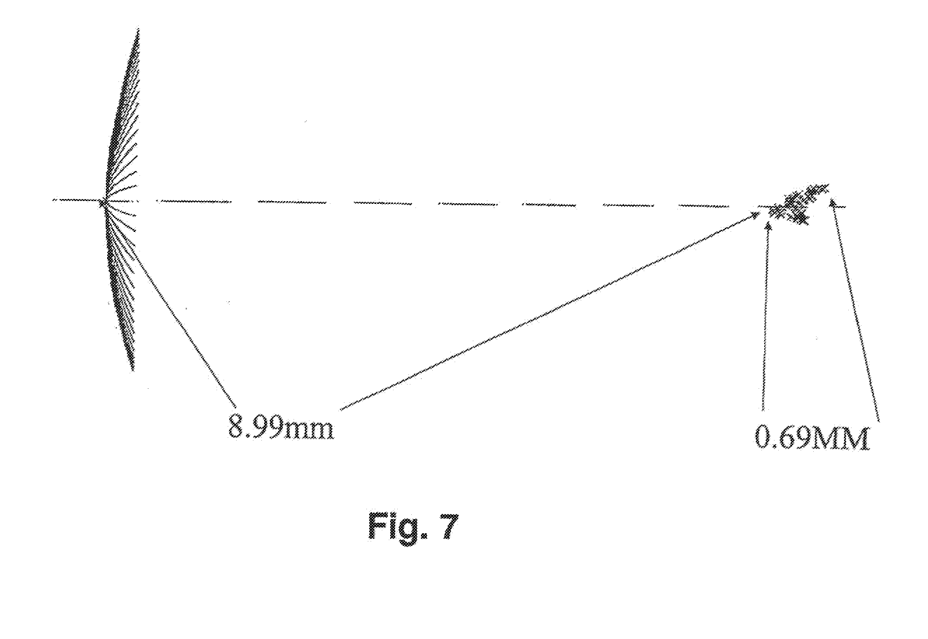

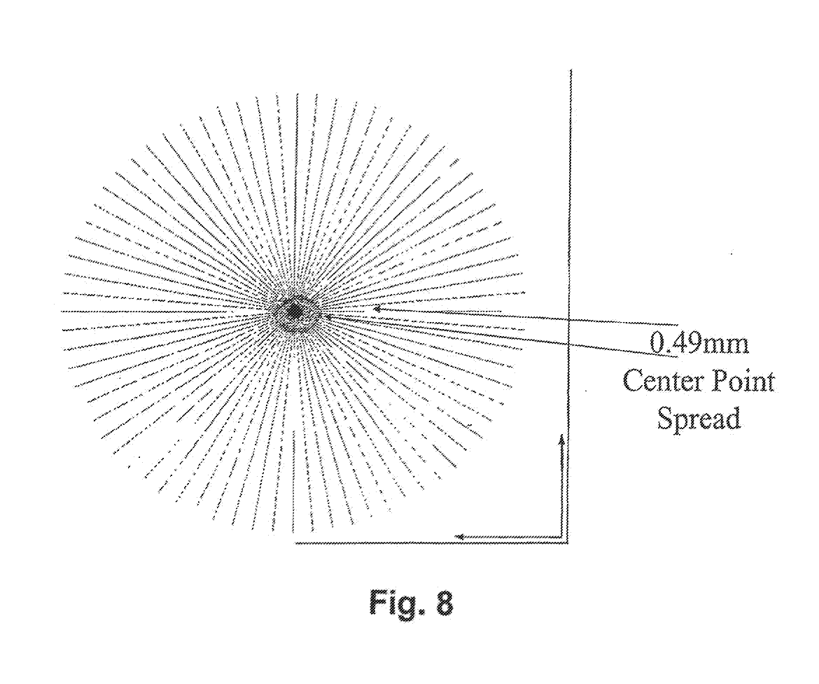

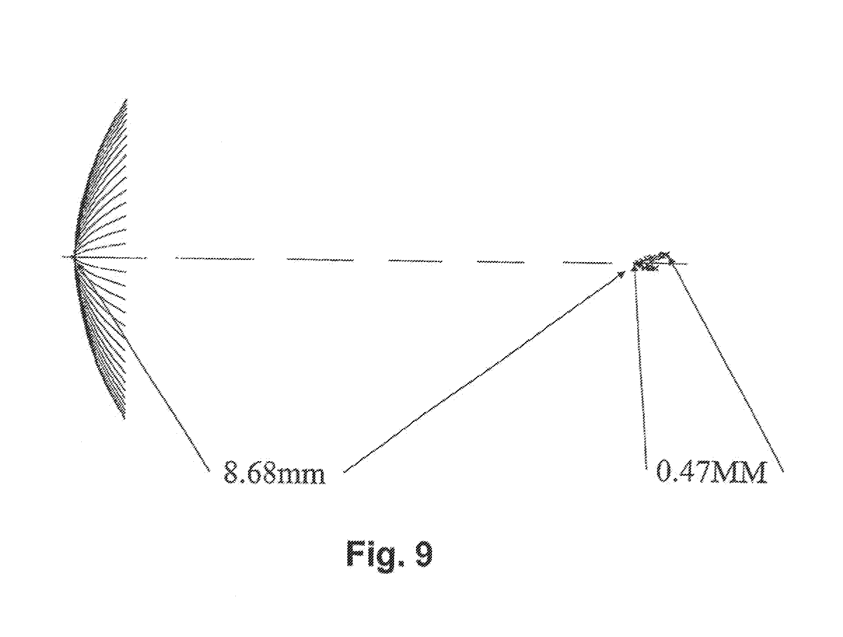

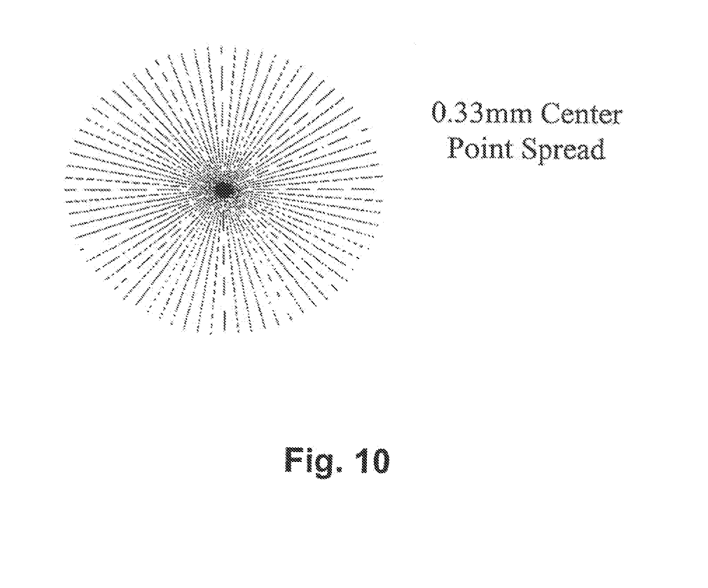

FIG. 5 is a focus scatter diagram along the LOCAL Z-AXIS for that portion of the cornea extending out to a 3.0 mm diameter. In this case, the focal points start at 7.06 mm along the LOCAL Z-AXIS and extend out an additional 6.91 mm. FIG. 6 illustrates that the radial scatter within a 3 mm diameter is 1.2 mm. Similarly, FIG. 7 illustrates that the axial focus scatter of a 5 mm diameter portion of the cornea begins at 8.99 mm and extends for an additional 1.69 mm. As shown in FIG. 8, the radial scatter of the same portion of the cornea is 0.49 mm. FIG. 9 illustrates that the axial focus scatter at 7 mm begins at 8.68 mm and extends axially for an additional 0.47 mm, whereas FIG. 10 illustrates that the corresponding radial scatter is 0.33 mm. Clearly, focus scatter is most severe in the central portion of the cornea, and decreases significantly as larger portions of the cornea are considered.

Therefore, it would clearly be desirable to reduce or eliminate the focus scatter at least in central portions of the cornea. However, for the purpose of relieving macular degeneration, it must not be eliminated entirely, but must be closely controlled.

In accordance with the present invention, this is accomplished by “orthogonalizing” at least a portion of the cornea. The term “orthogonalizing” refers to a re-shaping of the surface model so as to piecewise re-focus the cornea towards the LOCAL Z-AXIS. The re-shaped surface model can then be applied to the cornea (e.g. through ablation) or to shape the posterior surface of a contact lens (or another type of optical lens) so as to achieve the required focus scatter correction. It has been found that orthogonalizing the cornea not only reduces radial focus scatter, but simultaneously reduces axial focus scatter substantially and produces more uniformity in the radius of curvature of the orthogonalized portion of the cornea.

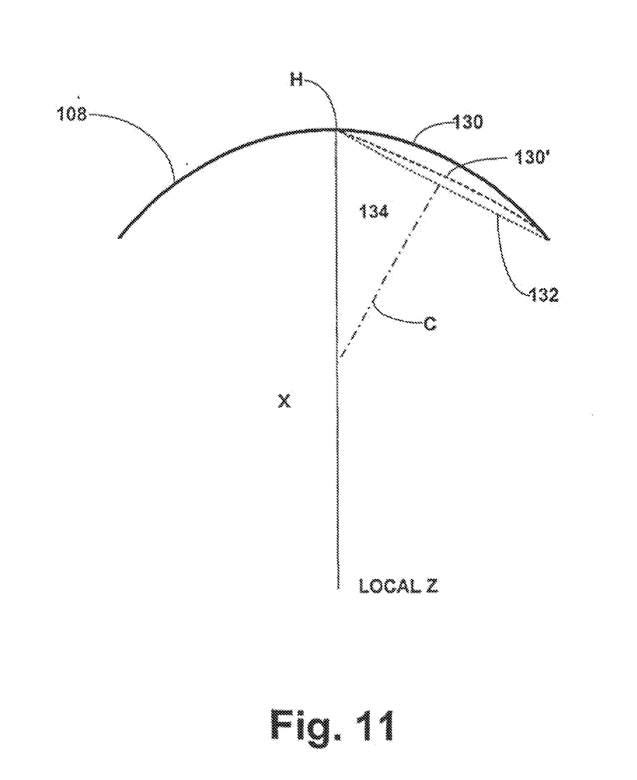

FIG. 11 illustrates the process of orthogonalization. The process is carried out on each of the arcs which represent characteristic curves, in the manner explained below. After this piecewise refocusing, the modified arcs are reassembled into a modified surface model having the refocused characteristics.

In FIG. 11, 130 represents one of the half-meridian arcs corresponding to a characterizing curve. Arc 130 has a center point C, the location of which has been exaggerated to demonstrate focus which is radially spaced from the LOCAL Z-AXIS. Orthogonalization of arc 130 begins with creating a chord 132 between the two ends of the arc. A perpendicular bisector 134 of chord 132 may be constructed, and it will pass through point C and intersect the LOCAL Z-AXIS at a point X. Using the distance of point X from point H (the HIGH point) as a radius, a new arc 130′ can now be drawn between the two end points of arc 130. Arc 130′ will be focused on the LOCAL Z-AXIS and will have a larger radius of curvature than arc 130.

At this point, arc 130′ could be accepted as an arc defining the modified surface model 108′. However, it would be desirable to avoid too great a change in the thickness of the cornea. Accordingly, a certain threshold is defined (for example 0.0075 mm), and if any portion of arc 130′ is more than a distance inside or outside the surface 108, arch 130′ is not accepted for use in the modified surface model. Instead, point x can be moved up or down on the LOCAL Z-AXIS (depending upon which direction arch 130′ needs to be moved) by half the excess over. Arc 130′ can then be re-drawn and re-tested against the threshold. This readjustment and testing continues until an acceptable arc 130′ has been found. Then, the next arc is orthogonalized. After all of the arcs are orthogonalized, a new surface model 108′ is created based upon all of the arcs.

As has been explained above, the orthogonalization process is applicable to corneal ablation procedures. Prior to the procedure, a corrected corneal surface model is generated, which is shaped to provide relief from macular degeneration and correction of refraction established by an eye test (as described in the patents cited above), and all the arcs are orthogonalized. The corrected corneal surface model is then registered with the unmodified corneal surface model, and it is moved towards the unmodified surface until the corrected surface just contacts the unmodified surface. If the point of initial contact is at the center of the corrected surface, it is moved toward the uncorrected surface until the periphery of the corrected surface just contacts the uncorrected surface. If the point of initial contact is at the periphery of the corrected surface, it is moved toward the uncorrected surface until the center of the corrected surface just contacts the uncorrected surface. The corrected surface will then be displaced so that it is, at least partially, inside the cornea, and the cornea is ablated until the displaced corrected surface becomes its new surface.

This procedure can be expected to reduce substantially the amount of material removed from the cornea, in comparison to all prior ablation techniques.

The central region of the retina is called the macula, and the very center of the macula, called the foveola, is the most sensitive. The macula typically has a diameter in the range of 6 to 7 millimeters, and the foveola typically has a diameter of about 0.35 mm. With perfect orthogonalization, all sub-portions of the cornea are refocused to the center of the macula, the foveola. However, this is the area usually affected by macular degeneration first, so it becomes necessary to spread the focus points away from the foveola while still controlling them. When orthogonalization is performed by refocusing all of the sub-regions onto the LOCAL Z-AXIS, orthogonalization is not perfect. The sub-portions of the cornea still focus on different points of the macula; some relief from macular degeneration is achieved. However, further adjustment of orthogonalization appears to be necessary in order to compensate effectively for macular degeneration.

In accordance with the present invention, sub-portions of the cornea are refocused so as to place their focal points outside the foveola yet still within the macula at a controlled distance from the LOCAL Z-AXIS. The macula has approximately the shape of a cap-shaped segment of a sphere, is usually between 6 millimeter and 7 millimeters in diameter and is approximately 0.88 millimeters deep. Optimum correction for macular degeneration is achieved when all sub-portions of the cornea are focused so as to make use of portions of the macula which are not affected by macular degeneration.

The difference should be kept in mind between introducing de-focus and the decentered focus of the invention. Ophthalmologists have long known that, in prescribing corrective lenses, distance focus can be reduced through de-focus, and a benefit in near vision can result. In accordance with the present invention, there is no de-focus. All sub-portions of the cornea are fully focused, but the focus point is moved away from an axis passing through the foveola, thereby achieving correction for macular degeneration.

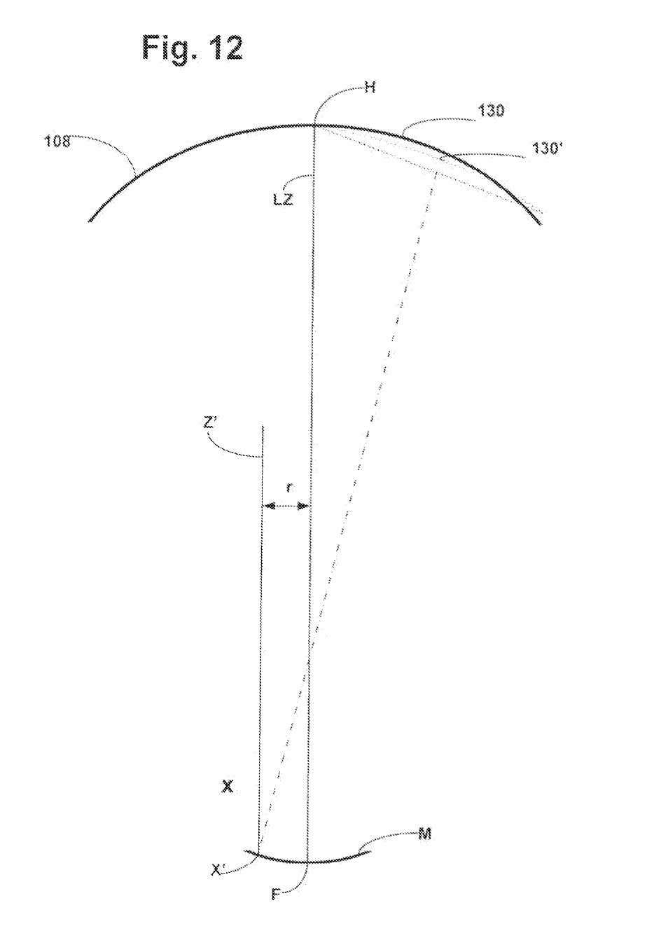

FIG. 12 illustrates the concept of decentered orthogonalization. The arc 130 is a sub-portion of the cornea which has a scattered focal point X. Ordinary orthogonalization as shown in FIG. 11 would move the focal point X to the LOCAL Z-AXIS, LZ. Perfect orthogonalization would move it to the foveola F on the macula M. Decentered orthogonalization creates a new arc 130′″ which focuses at a point X′, which is at a predefined radius r from the foveola. The axis Z′ is parallel to the LOCAL Z-AXIS and passes through the point X. For purposes of estimation, the macula can be considered flat in the region between the axes LZ and Z′.

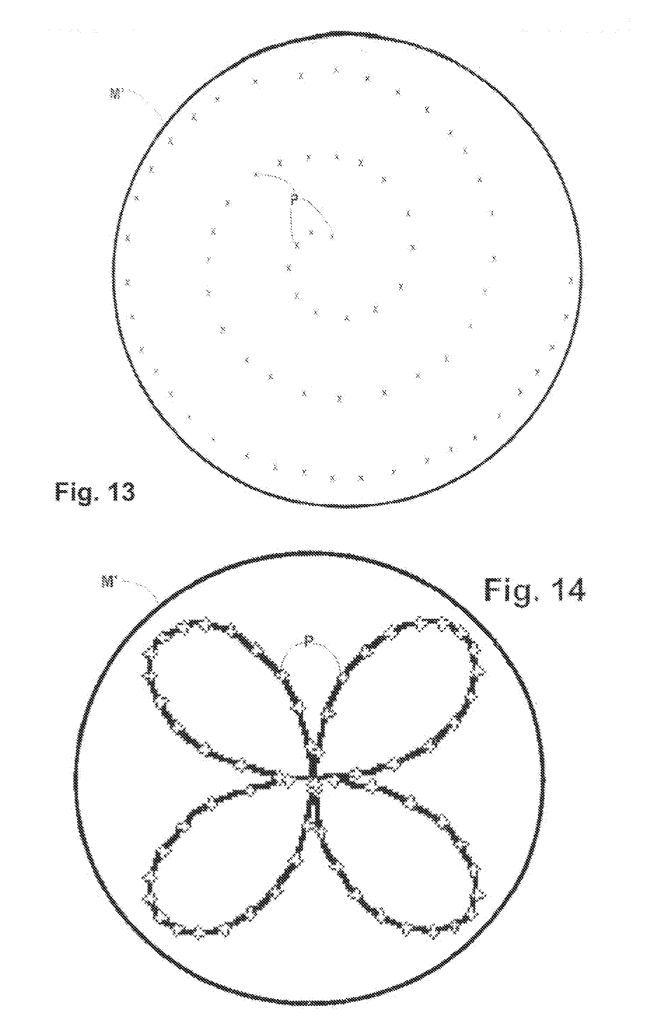

The preferred manner of performing decentered orthogonalization utilizes the technique discussed with respect to FIG. 4. Specifically, the anterior surface of the cornea is broken down into 72 arcs spaced 5° apart rotationally, and each arc is subjected to decentered orthogonalization. In order to achieve effective correction for macular degeneration, the 72 resulting focus points should be well distributed in a working region W′ of the foveola which preferably has a diameter less than 0.07 millimeters. FIG. 13 is a top plan view of the foveola showing the 72 points P distributed in a spiral pattern on the surface of the foveola.

A more preferred configuration for the points is illustrated in FIG. 14. This pattern is described by the polar equation R=a·cos 2e, where R is the two-dimensional radius of the point from the foveola, a is a constant selected to spread the points well over the entire working area M′, and e is the rotational angle of the particular arc on the cornea. This pattern is preferred to the spiral, because every quadrant of the working area M′ has focus points at a full range of distances from the foveola.

Another preferred pattern for the focus point is illustrated in FIG. 14. In this case, the pattern is formed from two overlaid rose patterns, a large one 150 and a small one 150′, which is offset by 45° from the pattern 150. Only one petal of each rose pattern is shown to have points, but it will be understood that each of the other petals is similarly provided with points. The points are shared evenly between the patterns 150 and 150′. However, the pattern 150 provides the outermost points and has points distributed at over its outermost two-thirds. Pattern 150′ provides the innermost points and has them evenly distributed. As a result, the pattern in FIG. 14 provides a good distribution of points near to and distant from the foveola.

It should be appreciated that, in all the focus point patterns that have been shown, in most instances the points are equally spaced along a curve. However, those skilled in the art will appreciate that unequal spacing could be provided for the points so as to concentrate them more in a specific region (e.g. the center or the outermost area of the working region.

A further method, defining a further embodiment of the invention, has been developed for decentered orthogonalization which is preferred over all those described previously for dealing with the effects of macular degeneration. The method proceeds exactly as in the FIG. 11, except that once arc 130′ has been reshaped, it is tilted clockwise so as to move the point X, the endpoint of the arc's axis, to the left, across the local z-axis so that it lies at a preselected distance from the local z-axis. At present, the preferred distance is approximately 0.01 mm. However, distances in the range of approximately 0.0025 mm to approximately 0.01 mm would still be effective to overcome the effects of macular degeneration.

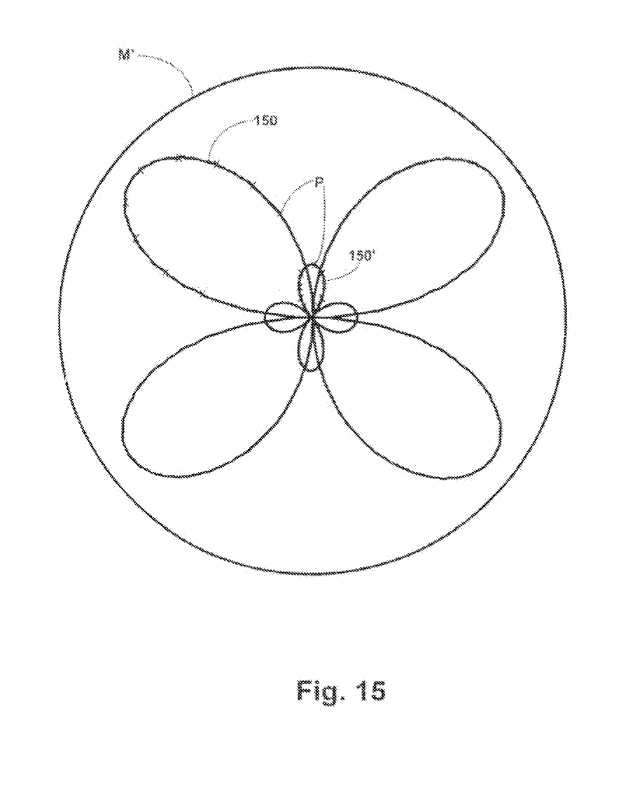

In accordance with yet a further embodiment, the lens may be constructed as explained with respect to any of FIGS. 11-15, and so that its position relative to the cornea is rotated circumferentially so as to tilt the local z-axis relative to the position shown and FIGS. 11 and 12. Preferably, the tilt of this axis is less than approximately 5°. Modern analysis methods permit an ophthalmologist to determine those areas of the macula which remain functional. After making such a determination, the lens construction orientation is modified, as explained above, so that local z-axis is tilted sufficiently to move the image produced by the lens off-center and onto a functional portion of the macula. The computer aided design system 630 (FIG. 1) can achieve such rotation of the entire structure by methods that are well-known.

Although preferred embodiments of the invention have been disclosed for illustrative purposes, those skilled in the art will appreciate that many additions, modifications, and substitutions are possible without departing from the scope and spirit of the invention. For example, the present invention is applicable not only to corneal ablation and contact lenses, but to any other kind of lens, including cataract, phakic, intraocular, intracorneal and spectacle lenses.

1. In a method for improving or planning the improvement of the vision of an eye, the steps of, on a surface model of the cornea of the eye, determining points of focus for different locations on the surface model and modifying the model so as to shift points of focus toward, but at a distance from, a predefined reference axis so as to place a plurality of points of focus in a pattern on the retina of the eye which is away from the reference axis, the modified model representing a desired restructuring of the cornea.

2. The method of claim 1 wherein the modifying step is representative of effectively re-shaping modeled cornea by one of physically changing its shape and applying to the eye an optical lens intended to change its refractive properties.

3. The method of claim 1, wherein the modifying step corresponds to conforming the shape of at least a portion of a surface of an optical lens to the modified surface model.

4. The method of claim 2 wherein physical changing comprises a possible corneal ablation of the modeled cornea.

5. The method of claim 2 wherein the optical lens is one of a contact lens, a cataract lens, a phakic lens an intraocular lens, an intracorneal lens and a spectacle lens.

6. The method of any one of claims 1-5 wherein the reference axis passes through the HIGH point.

7. The method of any one of claims 1-5 wherein the reference axis is the LOCALZ-AXIS.

8. The method of any one of claims 1-5 performed with the aid of computer program which produces the surface model of the cornea, which closely represents at least a portion of the surface of a cornea in three dimensions as a smooth, free-form surface, the modifying step comprising changing the shape of at least a portion of the model to produce a modified surface model.

9. The method of claims 1 wherein the pattern is a predefined pattern on the retina of the eye.

10. The method of claim 8 wherein the predetermined pattern is one of a circle, a spiral, a rose pattern and a dual rose pattern.

11. An optical lens for improving the vision of an eye, the lens comprising areas of focus on a surface thereof corresponding to different locations on the corneal surface of the eye, each area of focus being shaped to shift the focus of the corresponding location of the cornea toward, but at a distance from, a predefined reference axis so as to place a plurality of points of focus in a pattern on the retina of an eye containing the lens, which pattern is away from the reference axis.

12. The lens of claim 11 wherein the lens comprises one of a cataract lens, a phakic lens an intraoccular lens, an intracorneal lens and a spectacle lens.

13. The lens of any one of claim 11 or 12 wherein the reference axis passes through the HIGH point.

14. The lens of any one of claims 11 or 12 wherein the reference axis is the LOCALZ-AXIS.

15. The lens of any one of claims 11 or 12 designed with the aid of computer program which produces a surface model of the cornea which closely represents at least a portion of the surface of a cornea in three dimensions as a smooth, free-form surface, the model being modified in shape at each corresponding location at least a portion of the lens conforming in shape to the modified surface model.

16. In a system for improving the vision of an eye by effectively reshaping the cornea by one of controlling physically changing the shape of the cornea and controlling the shape of a lens to be applied to the eye to correct vision, a controller which controls said reshaping so as to shift points of focus for different locations on the surface of the cornea toward, but at a distance from, a predefined reference axis so as to place a plurality of points of focus in a pattern on the retina of the eye which is away from the reference axis.

17. The system of claim 16 wherein the lens comprises one of a cataract lens, a phakic lens an intraoccular lens, an intracorneal lens and a spectacle lens.

18. The system of any one of claim 16 or 17 wherein the controller causes reference axis to pass through the HIGH point.

19. The system of any one of claim 16 or 17 wherein the controller causes the reference axis to be substantially coincident with the LOCAL Z-AXIS.

20. The system of any one of claim 16 or 17 wherein the controller makes use of computer program which produces a surface model of the cornea which closely represents at least a portion of the surface of the cornea in three dimensions as a smooth, free-form surface, the controller causing the model to be modified in shape at each corresponding location so that at least a portion of the lens conforms in shape to the modified surface model.
